(19) United States
(12) Patent Application Publication
(10) Pub. No.: US 2022/0184017 A1
(43) Pub. Date: Jun. 16, 2022

(54) COMPOSITIONS FOR IMPROVING EYE HEALTH AND METHODS OF MAKING AND USING THEREOF

(71) Applicant: Oasis Medical, Inc., Glendora, CA (US)

(72) Inventor: James Marlow Christensen, Glendora, CA (US)

(21) Appl. No.: 17/685,171

(22) Filed: Mar. 2, 2022

Related U.S. Application Data
(63) Continuation of application No. PCT/US2020/050994, filed on Sep. 16, 2020.

(51) Int. Cl. A61K 31/202; A61K 36/185; A61K 31/047; A61K 31/122; A61K 9/00; A61K 9/48; A61P 27/04

(52) U.S. Cl. CPC A61K 31/202; A61K 36/185; A61K 31/047; A61P 27/04; A61K 9/0053; A61K 9/48; A61K 31/122

(57) ABSTRACT

Some embodiments disclosed herein relate to a composition containing an effective amount of dietary nutrients. In some embodiments, ingestion of the composition provides one or more of the following benefits, or others, improved functioning of an environmentally stressed eye, increased tear flow from the lacrimal glands, improved quality of the lipid secretion from the meibomian glands (e.g., to improve tear breakup time), and augmented DHA fatty acid in the retina damaged by blue light. In some embodiments, the composition is configured to scavenge free radicals in the eye to prevent premature degradation of the visual functions.

### CROSS-REFERENCE TO RELATED APPLICATIONS

This patent application is a continuation of International PCT Application No. PCT/US2020/050994, filed Sep. 16, 2020, which claims the benefit of priority to U.S. Provisional Patent Application No. 62/902,696, filed Sep. 19, 2019. The foregoing applications are fully incorporated herein by reference in their entireties for all purposes.

### FIELD OF THE INVENTION

Some embodiments herein pertain to compositions for use in methods of improving eye health and methods of making the same.

### BACKGROUND

Several disorders and diseases may affect the eye. For instance, dry eye is a condition in which a person lacks enough quality tears to lubricate and nourish the eye. Tears are necessary for maintaining the health of the front surface of the eye and for providing clear vision. Dry eye is a common and often chronic problem, particularly in older adults. Macular degeneration is another eye disorder and is the leading cause of severe, irreversible vision loss in people over age 60. It occurs when the small central portion of the retina, known as the macula, deteriorates. The retina is the light-sensing nerve tissue at the back of the eye. Because the disease develops as a person ages, it is often referred to as age-related macular degeneration (AMD). Although macular degeneration is almost never a totally blinding condition, it can be a source of significant visual disability.

### SUMMARY

Some embodiments disclosed herein provide unique compositions and formulations comprising a combination of nutrients that help to supplement and/or replace naturally present agents in the eye. In some embodiments, these combinations lead to improved eye health. In some embodiments, compositions as disclosed herein are configured to protect sensitive tissues (e.g., eye tissues) from damaging blue light. In some embodiments, compositions as disclosed herein are configured to neutralize free radicals that may damage tissues (e.g., in the eye) and vision. In some embodiments, compositions as disclosed herein may comprise docosahexaenoic acid (DHA) omega-3 fatty acid. In some embodiments, the composition comprises one or more of the following in any combination: DHA omega-3 fatty acid, a carotenoid, and/or an anthocyanins. In some embodiments, the composition comprises one or more of the following in any combination: DHA omega-3 fatty acid, lutein, zeaxanthin, astaxanthin, and/or an anthocyanin. In some embodiments, the composition comprises one or more of the following in any combination: DHA omega-3 fatty acid, lutein, zeaxanthin, astaxanthin, and/or an anthocyanin from the maqui berry.

Some embodiments pertain to a composition for improving eye health or function or for treating an environmentally stressed eye of a subject.

Any of the embodiments described above, or described elsewhere herein, can include or lack one or more of the following features.

In some embodiments, the composition comprises one or more omega-3 fatty acids. In some embodiments, the composition comprises a maqui berry extract. In some embodiments, the composition comprises one or more carotenoids.

In some embodiments, the one or more omega-3 fatty acids comprise DHA. In some embodiments, the omega-3 fatty acid comprises one or more of alpha-linolenic acid (ALA), docosapentaenic acid (DPA), and/or eicosapentaenoic acid (EPA). In some embodiments, the one or more omega-3 fatty acids have been enriched for DHA. In some embodiments, the one or more omega-3 fatty acids consists of or consists essentially of DHA. In some embodiments, the DHA is present in the composition in an amount ranging from 100 mg to 3000 mg.

In some embodiments, the maqui berry extract is present in an amount ranging from 30 mg to 200 mg. In some embodiments, the maqui berry extract comprises one or more anthocyanins. The anthocyanins may be composed of anthocyanadins and/or delpinidins.

In some embodiments, the one or more carotenoids comprises lutein, zeaxanthin, and/or astaxanthin. In some embodiments, lutein is present in an amount ranging from 6 mg to 40 mg. In some embodiments, the zeaxanthin is present in an amount ranging from 0.86 mg to 17 mg. In some embodiments, the astaxanthin is present in an amount ranging from 2 mg to 18 mg.

In some embodiments, the one or more omega-3 fatty acids, maqui berry extract, and one or more carotenoids is provided in an effective amount. In some embodiments, each of the one or more omega-3 fatty acids, DHA, maqui berry extract, lutein, zeaxanthin, and astaxanthin is provided in an effective amount.

In some embodiments, the composition is formulated for oral ingestion. In some embodiments, the composition is provided as a soft gel capsule.

Some embodiments pertain to a method of preventing, ameliorating, or treating an eye condition. In some embodiments, the method comprises administering a composition as disclosed elsewhere herein to a subject at risk for or suffering from an eye condition. In some embodiments, the eye condition comprises macular degeneration. In some embodiments, the eye condition comprises dry eye. In some embodiments, the eye condition comprises tired and fatigued eyes. In some embodiments, the eye condition is caused by environmental stress to the eye. In some embodiments, the method comprises administering the composition to a subject having an environmentally stressed eye.

Some embodiments pertain to a method of preparing the composition as disclosed elsewhere herein. In some embodiments, the method comprises mixing one or more omega-3 fatty acids with a maqui berry extract and one or more carotenoids to provide a mixture of ingredients. In some embodiments, the method comprises adding the mixture of ingredients to a soft gel. In some embodiments, the method comprises adding the mixture to a gummy gel or tablet.

Some embodiments pertain to an orally ingested composition for improving the functioning of an environmentally stressed eye comprising: effective amounts of omega-3 fatty acids, maqui berry extract, lutein, zeaxanthin, and astaxanthin. In some embodiments, the composition comprises 100-3000 mg DHA omega-3 fatty acids, 30-200 mg maqui berry extract, 6-40 mg lutein, 0.86-17 mg zeaxanthin, and 2-18 mg astaxanthin.

### BRIEF DESCRIPTION OF THE DRAWINGS

FIGS. 1A and 1B provide labels for softgels for oral administration comprising embodiments of compositions as disclosed herein.

### DETAILED DESCRIPTION

Embodiments of the disclosure relate to compositions for the treatment of an eye. Some embodiments disclosed herein pertain to composition for improving the quality of an eye affected by environmental stresses. In some embodiments, the composition comprises one or more omega-3 fatty acids, a maqui berry extract, and one or more carotenoids. In some embodiments, ingestion or administration of these combinations lead to improved eye health in a subject. In some embodiments, compositions as disclosed herein are configured to protect sensitive tissues (e.g., eye tissues) from damaging blue light. In some embodiments, compositions as disclosed herein are configured to neutralize free radicals that may damage tissues (e.g., in the eye) and vision. In some embodiments, compositions as disclosed herein are configured to improve tear flow and composition on the eye by improving the aqueous and lipid components of the eye. A variety of compositions are described below to illustrate various examples that may be employed to achieve one or more desired improvements. These examples are only illustrative and not intended in any way to restrict the general inventions presented and the various aspects and features of these inventions. Furthermore, the phraseology and terminology used herein is for the purpose of description and should not be regarded as limiting. No features or step disclosed herein is essential or indispensable.

### Definitions

Unless defined otherwise, all technical and scientific terms used herein have the same meaning as is commonly understood by one of ordinary skill in the art to which this disclosure belongs. All patents, applications, published applications, and other publications are incorporated by reference in their entirety. In the event that there is a plurality of definitions for a term herein, those in this section prevail unless stated otherwise.

As used herein, a “subject” refers to an animal that is the object of treatment, observation or experiment. “Animal” includes cold- and warm-blooded vertebrates and invertebrates such as fish, shellfish, reptiles, and, in particular, mammals. “Mammal” includes, without limitation, mice, rats, rabbits, guinea pigs, dogs, cats, sheep, goats, cows, horses, primates, such as monkeys, chimpanzees, apes, and, in particular, humans. In some embodiments, the subject can be human. In some embodiments, the subject can be a child and/or an infant. In other embodiments, the subject can be an adult. In some embodiments, the adult is over the age of 60.

As used herein, the terms “treat,” “treating,” “treatment,” “therapeutic,” and “therapy” can but does not necessarily mean curing or abolition of the disease or condition. Any alleviation of any undesired signs or symptoms of the disease or condition, to any extent can be considered treatment and/or therapy. The term “prophylactic treatment” refers to treating a subject who does not yet exhibit symptoms of a disease or condition, but who is susceptible to, or otherwise at risk of, a particular disease or condition, whereby the treatment reduces the likelihood that the patient will develop the disease or condition. The term “therapeutic treatment” also refers to administering treatment to a subject already suffering from a disease or condition.

The terms “therapeutically effective amount” and “effective amount” are used to indicate an amount of an active compound, or pharmaceutical agent, that elicits the biological or medicinal response indicated. For example, a therapeutically effective amount of compound, salt or composition can be the amount needed to prevent, alleviate or ameliorate symptoms of the disease or condition of the subject being treated. This response may occur in a tissue, system, animal, or human and includes alleviation of the signs or symptoms of the disease or condition being treated. Determination of an effective amount is well within the capability of those skilled in the art, in view of the disclosure provided herein. The therapeutically effective amount of the compounds disclosed herein required as a dose will depend on the route of administration, the type of animal, including human, being treated and the physical characteristics of the specific animal under consideration. The dose can be tailored to achieve a desired effect, but will depend on such factors as weight, diet, concurrent medication and other factors which those skilled in the medical arts will recognize.

The term “pharmaceutical composition” refers to a mixture of one or more compounds and/or salts disclosed herein with other chemical components, such as diluents or carriers. The pharmaceutical composition facilitates administration of the compound to an organism. Pharmaceutical compositions can also be obtained by reacting compounds with inorganic or organic acids such as hydrochloric acid, hydrobromic acid, sulfuric acid, nitric acid, phosphoric acid, methanesulfonic acid, ethanesulfonic acid, p-toluenesulfonic acid and salicylic acid. Pharmaceutical compositions will generally be tailored to the specific intended route of administration.

The term “physiologically acceptable” defines a carrier, diluent or excipient that does not abrogate the biological activity and properties of the compound nor cause appreciable damage or injury to an animal to which delivery of the composition is intended.

As used herein, a “carrier” refers to a compound that facilitates the incorporation of a compound into cells or tissues. For example, without limitation, dimethyl sulfoxide (DMSO) is a commonly utilized carrier that facilitates the uptake of many organic compounds into cells or tissues of a subject.

As used herein, a “diluent” refers to an ingredient in a pharmaceutical composition that lacks appreciable pharmacological activity but may be pharmaceutically necessary or desirable. For example, a diluent may be used to increase the bulk of a potent drug whose mass is too small for manufacture and/or administration. It may also be a liquid for the dissolution of a drug to be administered by injection, ingestion or inhalation. A common form of diluent in the art is a buffered aqueous solution such as, without limitation, phosphate buffered saline that mimics the pH and isotonicity of human blood.

As used herein, an “excipient” refers to an essentially inert substance that is added to a pharmaceutical composition to provide, without limitation, bulk, consistency, stability, binding ability, lubrication, disintegrating ability etc., to the composition. For example, stabilizers such as anti-oxidants and metal-chelating agents are excipients. In an embodiment, the pharmaceutical composition comprises an anti-oxidant and/or a metal-chelating agent. A “diluent” is a type of excipient.

As used herein, the term “weight percent,” when referring to a component, is the weight of the component divided by the weight of the composition that includes the component, multiplied by 100%. For example, the weight percent of component A when 5 grams of component A is added to 95 grams of component B is 5% (e.g., 5 g A/(5 g A+95 g B)×100%).

As used herein, “kit” means a collection of at least two components constituting the kit. Together, the components constitute a functional unit for a given purpose. Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation.

As used herein, “instruction(s)” means documents describing relevant materials or methodologies pertaining to a kit. These materials may include any combination of the following: background information, list of components and their availability information (purchase information, etc.), brief or detailed protocols for using the kit, trouble-shooting, references, technical support, and any other related documents. Instructions can be supplied with the kit or as a separate member component, either as a paper form or an electronic form which may be supplied on computer readable memory device or downloaded from an internet website, or as recorded presentation. Instructions can comprise one or multiple documents, and are meant to include future updates.

Terms and phrases used in this application, and variations thereof, especially in the appended claims, unless otherwise expressly stated, should be construed as open ended as opposed to limiting. As examples of the foregoing, the term “including” should be read to mean “including, without limitation,” “including but not limited to,” or the like; the term “comprising” as used herein is synonymous with “including,” “containing,” or “characterized by,” and is inclusive or open-ended and does not exclude additional, unrecited elements or method steps; the term “having” should be interpreted as “having at least;” the term “includes” should be interpreted as “includes but is not limited to;” the term “example” is used to provide exemplary instances of the item in discussion, not an exhaustive or limiting list thereof; and use of terms like “preferably,” “preferred,” “desired,” or “desirable,” and words of similar meaning should not be understood as implying that certain features are critical, essential, or even important to the structure or function of the invention, but instead as merely intended to highlight alternative or additional features that may or may not be utilized in a particular embodiment of the invention. In addition, the term “comprising” is to be interpreted synonymously with the phrases “having at least” or “including at least”. When used in the context of a process, the term “comprising” means that the process includes at least the recited steps, but may include additional steps. When used in the context of a compound, composition or device, the term “comprising” means that the compound, composition or device includes at least the recited features or components, but may also include additional features or components. Likewise, a group of items linked with the conjunction ‘and’ should not be read as requiring that each and every one of those items be present in the grouping, but rather should be read as ‘and/or’ unless expressly stated otherwise. Similarly, a group of items linked with the conjunction ‘or’ should not be read as requiring mutual exclusivity among that group, but rather should be read as ‘and/or’ unless expressly stated otherwise. Additionally, the phrase “consisting essentially of” will be understood to include those elements specifically recited and those additional elements that do not materially affect the basic and novel characteristics of the claimed technology. The phrase “consisting of” excludes any element not specified.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article “a” or “an” does not exclude a plurality. A single processor or other unit may fulfill the functions of several items recited in the claims. The mere fact that certain measures are recited in mutually different dependent claims does not indicate that a combination of these measures cannot be used to advantage. Any reference signs in the claims should not be construed as limiting the scope.

### Compositions

Described herein are formulations and compositions comprising one or more compounds (e.g., active agents, active ingredients, etc.). Some embodiments described herein relate to a composition that can include a therapeutically effective amount of one or more compounds described herein. In some embodiments, the composition may comprise one or more omega-3 fatty acids or esters thereof. In some embodiments, the composition may comprise a maqui berry extract. In some embodiments, the composition may comprise one or more carotenoids. In some embodiments, the composition may comprise one or more omega-3 fatty acids and a maqui berry extract. In some embodiments, the composition comprises one or more omega-3 fatty acids, a maqui berry extract, and one or more carotenoids. In some embodiments, the composition further comprises at least one or more excipient, carrier, or inert ingredient. In some embodiments, the one or more excipient, carrier, or inert ingredient is selected from gelatin, glycerin, purified water, beeswax (e.g., yellow), lecithin oil, and the like.

In some embodiments, the compositions as described herein combines an effective amount of dietary nutrients that can improve the functioning of an environmentally stressed eye. In some embodiments the compositions as described herein are configured to, among other things, scavenge free radicals in the eye to prevent premature degradation of the visual functions. In some embodiments, is the compositions as described herein may be administered to a subject to improve eye health.

Omega-3 essential fatty acids are structural components of all tissues and are indispensable for cell membrane synthesis. Docosahexaenoic acid (DHA) omega-3 fatty acid is naturally present in the retinal tissues and supports cell membrane synthesis. In the retina, DHA is mainly located in the photoreceptor cells, where it becomes esterified into phospholipids. It plays an important role in the synthesis of disk membranes, in providing an adequate environment for conformational rhodopsin changes, and in modifying the activity of retinal enzymes. Approximately, 60 molecules of phospholipids surround each molecule of visual pigment. A deficiency of DHA in the membranes of photoreceptors disturbs membrane fluidity and function and could alter the process of outer segment renewal. Omega-3 fatty acids also supports lipid layer synthesis in the meibomiam glands. The lipid layer is required for evaporation protection and stabilization of the ocular tear film. Clinical trials have found that doses of omega-3 fatty acid containing DHA ranging from 100 mg to 3000 mg per day can have clinically beneficial effects, including alleviating issues associated with deficiencies of DHA.

Additionally, the principal cause of dry eyes appears to be the dysfunction of the Meibomian gland, due to chronic inflammation. In some embodiments, the breakdown of omega-3 fatty acid in the body leads to a suppression of this inflammation. In the eyelids, inflammation damages the Meibum making it stiffer and more viscous, and less able to protect the tears and ocular surface resulting in dry eye. In some embodiments, the compositions as described herein may change the composition of the Meibum making it more fluid. In some embodiments, patients who receive 500 mg of omega-3 supplements may experience significant improvement of their dry eye symptoms, including significant increases in the tear breakup time. In some embodiments, the compositions as described herein may also lead to a reduction of blocked meibomian ducts and improvement in meibomitis.

In some embodiments, as disclosed elsewhere herein, the composition comprises one or more omega-3 fatty acids or esters thereof. For example, the omega-3 fatty acids or esters thereof are in a triglyceride form, re-esterified triglyceride concentrates, an ethyl ester form, a free fatty acid form, or a phospholipids form. In some embodiments, the omega-3 fatty acids or esters thereof are in triglyceride form. In some embodiments, the amount of the one or more omega-3 fatty acids present (in mg) per dose is equal to or greater than about: 20, 30, 40, 50, 75, 100, 125, 150, 175, 200, 225, 250, 300, 350, 400, 450, 500, 600, 700, 800, 900, 1000, 2000, 3000, 4000, or ranges including and/or spanning the aforementioned values. For example, the one or more omega-3 fatty acids present per dose may range from 200 mg to 300 mg, from 175 mg to 450 mg, from 150 mg to 300 mg, etc. In some embodiments, the omega-3 fatty acid comprises or consists of DHA. In some embodiments, the amount of DHA fatty acid present (in mg) per dose is equal to or greater than about: 0.1, 1, 10, 20, 30, 40, 50, 75, 100, 125, 150, 175, 200, 225, 250, 300, 350, 400, 450, 500, 600, 700, 800, 900, 1000, 2000, 3000, 4000, or ranges including and/or spanning the aforementioned values. For example, the DHA present per dose may range from 150 mg to 250 mg, from 175 mg to 225 mg, from 150 mg to 300 mg, etc. In some embodiments, the weight percent of omega-3 fatty acid present in the composition is equal to or greater than about: 20%, 40%, 50%, 60%, 70%, 80%, or ranges including and/or spanning the aforementioned values. For example, the weight percent of omega-3 fatty acid present in the composition may range from 20% to 60%, 40% to 60%, 10% to 70%, etc. In some embodiments, the weight percent of DHA present in the composition is equal to or greater than about: 10%, 20%, 40%, 50%, 60%, 70%, or ranges including and/or spanning the aforementioned values. For example, the weight percent of DHA present in the composition may range from 20% to 60%, 40% to 60%, 10% to 70%, etc. In some embodiments, the weight percent of DHA present in the one or more omega-3 fatty acids is equal to or greater than about: 50%, 60%, 70%, 80%, 85%, 90%, 95%, 99%, 100%, or ranges including and/or spanning the aforementioned values.

In some embodiments, as disclosed elsewhere herein, the composition may comprise DHA, ALA, DPA and/or EPA omega-3 fatty acids. In some embodiments, the composition lacks or substantially lacks ALA, DPA and/or EPA omega-3 fatty acids. In some embodiments, the amount of any one of ALA, DPA, and/or EPA present (in mg) per dose is less than or equal to about: 0.1, 1, 10, 20, 30, 40, 50, 75, 100, 125, 150, 175, 200, 225, 250, 300, 350, 400, 450, 500, 600, 700, 800, 900, 1000, or ranges including and/or spanning the aforementioned values. In some embodiments, the ratio of any two of DHA, ALA, DPA, or EPA (e.g., DHA:ALA, DHA:DPA, DHA:EPA, ALA:DPA, etc.) is at least or about 0.2:1, 0.25:1, 0.5:1, 0.7:1 0.75:1, 1:1, 1.25:1, 1.5:1, 1.75:1, 2:1, 2.25:1, 2.5:1, 2.75:1, 3:1, 3.25:1, 3.5:1, 3.75:1, 4:1, 4.25:1, 4.5:1, 4.75:1, 5:1, 5.25:1, 5.5:1, 5.75:1, 6:1, or ranges of ratios including and/or spanning the aforementioned ratios. In some embodiments, the ratio of any two of DHA, ALA, DPA, or EPA is about 0.2:1 to about 6:1, about 0.25:1 to about 5.75:1, about 5:1 to about 5.5:1, about 0.7:1 to about 5:1, about 0.75:1 to about 4.75:1, about 1:1 to about 4:1, or about 2:1 to about 3:1, or ranges of ratios including and/or spanning the aforementioned ratios. In some embodiments, the ratio of any two of DHA, ALA, DPA, or EPA is about 2:1 to about 3:1. In some embodiments, the ratio of two of DHA, ALA, DPA, or EPA is 2:1. In some embodiments, the ratio of two of DHA, ALA, DPA, or EPA is 1.5:1. In some embodiments, the ratio of DHA to the combination of DHA, ALA, DPA, and EPA is 1:2, 2:3, 4:5, or ranges of ratios including and/or spanning the aforementioned ratios.

Carotenoids such as lutein, zeaxanthin, and astaxanthin help protect the retina from oxidative damage initiated by the absorption of blue light. Lutein and zeaxanthin are structurally isomers of each other and are more effective when combined. They are the only carotenoids found in high concentrations in the macula and surrounding retinal tissues where they protect the eye by absorbing up to 90% of blue light reaching these critical visual structures of the eye. They protect the macula from light-induced oxidative damage and scavenge free radicals which can damage cells, contribute to aging, and lead to the progression of diseases like macular degeneration, senile cataracts, and decreased visual acuity. In some embodiments, these carotenoids can reduce headaches, eye strain, and eye fatigue by increasing contrast reduced by the effects of blue light on the retina.

Astaxanthin is another carotenoid. Unlike lutein and zeaxanthin which deposits in the back of the eye, astaxanthin deposits in the front of the eye, in the ciliary body. It is produced by the microalgae Haematococcus pluvialis under high stress conditions. It is a very powerful natural anti-inflammatory, has very strong free radical scavenging activity, and is far more effective than other carotenoids at singlet oxygen quenching. It protects rods, cones, and DHA containing membranes in the eye and is a potent inhibitor of DHA oxidation. It prevents light-induced damage, photoreceptor cell damage, ganglion cell damage, and damage to the neurons of the inner retinal layers. Astaxanthin also supports the ciliary body as they contract to focus the eyes by supporting muscle recovery and minimizing exercise-induce muscle damage. This leads to less eye fatigue, headaches, and shoulder and neck aches.

In some embodiments, the composition comprises one or more carotenoids. In some embodiments, the amount of the one or more carotenoids present (in mg) per dose is equal to or greater than about: 0.1, 0.5, 0.75, 1, 1.5, 2.0, 2.5, 3.0, 3.5, 4.0, 4.5, 5, 7.5, 10, 12.5, 15.0, 17.5, 20, 30, 40, 50, 75, 100, 125, 150, 175, 200, or ranges including and/or spanning the aforementioned values. For example, the carotenoids present per dose may range from 0.5 mg to about 3 mg, from 1 mg to about 3 mg, from 0.5 mg to 40 mg, from 10 mg to 20 mg, from 1 mg to 30 mg, from 1 mg to 35 mg, from 5 mg to 30 mg, from 19 mg to about 35 mg, etc. In some embodiments, the weight percent of the one or more carotenoids present in the composition is equal to or less than about: 2.5%, 5%, 7.5%, 10%, 12.5%, 15%, 20%, 25%, or ranges including and/or spanning the aforementioned values. For example, the weight percent of the one or more carotenoids present in the composition may range from 5% to 20%, 2.5% to 10%, 2.5% to 15%, etc. In some embodiments, the one or more carotenoids comprises or consists of one or more of lutein, zeaxanthin, and/or astaxanthin.

In some embodiments, the amount of lutein present (in mg) per dose is equal to or greater than about: 0.1, 1, 5, 6, 10, 20, 30, 40, 50, 75, 100, or ranges including and/or spanning the aforementioned values. For example, the lutein present per dose may range from 6 mg to 20 mg, from 1 mg to 20 mg, from 5 mg to 10 mg, etc. In some embodiments, lutein is present in an amount ranging from 6 mg to 40 mg. In some embodiments, the weight percent of lutein present in the composition is equal to or less than about: 2%, 5%, 7.5%, 10%, 15%, 20%, or ranges including and/or spanning the aforementioned values. For example, the weight percent of lutein present in the composition may range from 2% to 7.5%, 5% to 10%, 2% to 15%, etc.

In some embodiments, the amount of zeaxanthin present (in mg) per dose is equal to or greater than about: 0.1, 0.5, 0.75 1, 1.25, 1.5, 1.75, 2, 2.5, 5, 7.5, 10, 20, 30, 40, 50, or ranges including and/or spanning the aforementioned values. For example, the zeaxanthin present per dose may range from 1 mg to 5 mg, from 0.1 mg to 10 mg, from 1 mg to 2 mg, etc. In some embodiments, the zeaxanthin is present in an amount ranging from 0.86 mg to 17 mg. In some embodiments, the weight percent of zeaxanthin present in the composition is equal to or less than about: 0.1%, 1%, 2%, 5%, 10%, 15%, or ranges including and/or spanning the aforementioned values. For example, the weight percent of zeaxanthin present in the composition may range from 1% to 5%, 0.1% to 10%, 0.1% to 5%, etc.

In some embodiments, the amount of astaxanthin present (in mg) per dose is equal to or greater than about: 0.1, 1, 2, 5, 7.5, 10, 12.5, 15.0, 17.5, 20, 30, 40, 50, or ranges including and/or spanning the aforementioned values. For example, the astaxanthin present per dose may range from 1 mg to 5 mg, from 0.1 mg to 10 mg, from 1 mg to 2 mg, etc. In some embodiments, the astaxanthin is present in an amount ranging from 2 mg to 18 mg. In some embodiments, the weight percent of astaxanthin present in the composition is equal to or less than about: 0.1%, 1%, 2%, 5%, 10%, 15%, or ranges including and/or spanning the aforementioned values. For example, the weight percent of astaxanthin present in the composition may range from 1% to 5%, 0.1% to 10%, 0.1% to 5%, 0.1% to 2%, etc.

In some embodiments, doses of lutein ranging from 6 mg to 40 mg per day and zeaxanthin ranging from 0.86 mg to 17 mg per day may increase macular pigment density. In some embodiments, doses of astaxanthin ranging from 2 mg to 18 mg per day may have beneficial effects as described herein.

Maqui Berry (Aristotelia chilensis) is an antioxidant superfruit, containing high concentrations of polyphenols and anthocyanins. The anthocyanins from maqui berry extract are powerful antioxidants which support the health of the eye and circulatory system by providing antioxidant activity and support for aqueous tear fluid production in the lacrimal glands. The delphinidin anthocyanins reduce damage to photoreceptor cells caused by light stimulation, protect cells and membranes in the eye from free-radical damage, and support aqueous tear fluid production in lacrimal glands by eliminating the free radicals and reactive oxygen species. 30 mg to 200 mg of maqui berry extract daily, standardized to 10-35% total anthocyanins and 8-25% delphinidins aid in the management of lacrimal glands function in dry eye patients. A number of berries such as acai berries, blueberries, bilberries, raspberries, and goji berries are good sources of antioxidant polyphenols, called anthocyanins, and are effective in reducing inflammation and oxidative stress. Each type of berry has unique ingredients that can also have superior health benefits over the other berries in the above group. Maqui berries are also an excellent source of antioxidant polyphenols and the extract has been shown to offer health-promoting effects by reducing oxidative stress.

For example, a dose of 162 mg of a maqui berry extract three times daily can result in significant reduced blood measures of free radical damage. For another example, maqui berry extract can increase the flow of tears from the lachrymal glands of the eye. In some embodiments, taking 30 mg to 60 mg of a concentrated maqui berry extract each day may increase tear production by roughly 50%.

In some embodiments, as disclosed elsewhere herein, the composition comprises maqui berry extract. In some embodiments, the anthocyanins of the maqui berry as used. Exemplary anthocyanins of the maqui berry include, but are not limited to, delphinidin 3-sambubioside-5-glucoside; delphinidin 3 ,5-diglucoside; cyanidin 3-sambubioside-5-glucoside; cyanidin 3 ,5-diglucoside; delphinidin 3-sambubioside; delphinidin 3-glucoside; cyanidin 3-sambubioside; and cyanidin 3-glucoside. In some embodiments, the anthocyanins are isolated from the maqui berry. In some embodiments, the amount of the maqui berry extract present (in mg) per dose is equal to or greater than about: 20, 30, 40, 50, 75, 100, 125, 150, 175, 200, 225, 250, 300, 350, 400, 450, 500, 600, 700, 800, 900, 1000, 2000, or ranges including and/or spanning the aforementioned values. For example, the maqui berry extract present per dose may range from 50 mg to 100 mg, from 75 mg to 225 mg, from 30 to 200 mg, from 40 mg to 300 mg, etc. In some embodiments, the weight percent of maqui berry extract present in the composition is equal to or greater than about: 5%, 10%, 20%, 25%, 30%, 35%, 40%, 50%, 60%, 70%, or ranges including and/or spanning the aforementioned values. For example, the weight percent of maqui berry extract present in the composition may range from 5% to 30%, 20% to 35%, 10% to 30%, etc. In some embodiments, maqui berry extract comprises anthocyanins and delphinidins. In some embodiments, weight percent of anthocyanins in the maqui berry extract is less than or equal to: 1%, 2.5%, 5%, 10%, 20%, 25%, 30%, 35%, 45%, or ranges including and/or spanning the aforementioned values. In some embodiments, weight percent of delphinidins in the maqui berry extract is less than or equal to: 1%, 2.5%, 5%, 8%, 10%, 20%, 25%, 30%, 35%, 45%, or ranges including and/or spanning the aforementioned values. In some embodiments, the maqui berry extract is standardized to 10-35% total anthocyanins and 8-25% delphinidins.

In some embodiments, the improvement in function is measured as one or more of an increase in aqueous tear flow from the lacrimal glands as measured by the Schirmer test; improvement in the quality of the lipid secretion from the meibomian glands as measured by improvement of the tear breakup time; augmentation of the DHA fatty acid in the retina resulting from oxidative damage by blue light as measure by improved contrast sensitivity and visual acuity; and relief from eye strain, fatigue, and headaches caused by reduced stress and fatigue on the ciliary muscles, or other improvements (including as disclosed elsewhere herein).

In some embodiments, the composition includes one or more omega-3 fatty acids, maqui berry extract, and one or more carotenoids. In some embodiments, the composition does not include vitamins. In some embodiments, the composition does not include vitamin C. In some embodiments, the composition does not include zinc. In some embodiments, the composition does not include a pigment source. In some embodiments, the composition does not include copper. In some embodiments, the composition does not include selenium. In some embodiments, the composition does not include a ratio of the omega-3 fatty acids or esters thereof to the maqui berry extract from about 12:1 to about 150:1. In some embodiments, the one or more omega-3 fatty acids does not include a ratio of EPA to DHA in a range from 0.1:1 to 5:1 In some embodiments, the composition does not include hill oil. In some embodiments, the composition does not include about 50 to about 500 mg of krill oil. In some embodiments, the composition does not include ginkgo biloba. In some embodiments, the composition does not include quercetin. In some embodiments, the composition does not include resveratrol.

In some embodiments, the composition as described herein is administered to a subject in need thereof. In some embodiments, the composition is not adapted for transmucosal administration. In some embodiments, the composition is not administered as a chewing gum. In some embodiments, the composition described herein is not administered to treat ocular diseases selected from glaucoma, uvea disease or diabetic eye disease. In some embodiments, the composition administered does not include a ratio of the omega-3 fatty acids or esters thereof to the maqui berry extract from about 12:1 to about 150:1. In some embodiments, the one or more omega-3 fatty acids administered does not include a ratio of EPA to DHA in a range from 0.1:1 to 5:1. In some embodiments, the composition administered does not comprise hill oil. In some embodiments, the composition administered does not include about 50 to about 500 mg of krill oil.

In some embodiments, the composition consists of or consists essentially of one or more omega-3 fatty acids, maqui berry extract, and one or more carotenoids. In some embodiments, the omega-3 fatty acid comprises one or more of alpha-linolenic acid (ALA), docosapentaenic acid (DPA), and/or eicosapentaenoic acid (EPA). In some embodiments, the one or more omega-3 fatty acids have been enriched for DHA. In some embodiments, the maqui berry extract (e.g., aristotleia chilensis extract) comprises one or more anthocyanins. The anthocyanins may be composed of anthocyanadins and/or delpinidins. In some embodiments, the one or more carotenoids comprises lutein, zeaxanthin, and/or astaxanthin. In some embodiments, the composition consists of or consists essentially of one or more ALA, DPA, EPA, maqui berry extract, lutein, zeaxanthin, and astaxanthin.

### Pharmaceutical Compositions

Some embodiments described herein relate to a pharmaceutical composition (e.g., formulations), that can include an effective amount of one or more compounds described herein (for example, a compound of as disclosed herein, or a pharmaceutically acceptable salt thereof) and a pharmaceutically acceptable (e.g., physiologically acceptable) carrier, diluent, excipient or combination thereof.

The pharmaceutical compositions described herein can be administered to a human patient per se, or in pharmaceutical compositions where they are mixed with other active ingredients, as in combination therapy, or carriers, diluents, excipients or combinations thereof. Proper formulation is dependent upon the route of administration chosen. Techniques for formulation and administration of the compounds described herein are known to those skilled in the art.

Pharmaceutical compositions including one or more omega-3 fatty acids, a maqui berry extract, one or more carotenoids, and at least one excipient are provided. In some embodiments, administration of the pharmaceutical composition is formulated for oral formulations, however, other routes of administration are also contemplated.

The pharmaceutical compositions described herein can be administered by themselves to a subject, or in compositions where they are mixed with other active agents, as in combination therapy, or with carriers, diluents, excipients or combinations thereof. Formulation is dependent upon the route of administration chosen. Techniques for formulation and administration of the compounds described herein are known to those skilled in the art (see, e.g., “Remington: The Science and Practice of Pharmacy”, Lippincott Williams & Wilkins; 20th edition (Jun. 1, 2003) and “Remington's Pharmaceutical Sciences,” Mack Pub. Co.; 18th and 19th editions (December 1985, and June 1990, respectively).

The pharmaceutical compositions disclosed herein may be manufactured by a process that is itself known, e.g., by means of conventional mixing, dissolving, granulating, dragee-making, levigating, emulsifying, encapsulating, entrapping, tableting, or extracting processes. Many of the compounds used in the pharmaceutical combinations disclosed herein may be provided as salts with pharmaceutically acceptable counterions.

Multiple techniques of administering a compound exist in the art including, but not limited to, oral, rectal, topical, aerosol, injection and parenteral delivery, including intramuscular, subcutaneous, intravenous, intramedullary injections, intrathecal, direct intraventricular, intraperitoneal, intranasal and intraocular injections. Contemplated herein is any combination of the forgoing, or other methods as would be known to one of ordinary skill in the art (see, e.g., “Remington: The Science and Practice of Pharmacy”, Lippincott Williams & Wilkins; 20th edition (Jun. 1, 2003) and “Remington's Pharmaceutical Sciences,” Mack Pub. Co.; 18th and 19th editions (December 1985, and June 1990, respectively). In some embodiments, the composition described herein are administered to a subject by oral administration.

In some embodiments, a pharmaceutical composition described herein may be combined in intimate admixture with a pharmaceutical carrier according to and at least one excipient are provided conventional pharmaceutical compounding techniques. The carrier can take a wide variety of forms depending on the form of preparation desired for administration. Thus, the pharmaceutical compositions provided herein can be presented as discrete units suitable for oral administration such as capsules, cachets or tablets each containing a predetermined amount of one or more of the active ingredients. Further, the compositions can be presented as an oil, a powder, as granules, as a solution, as a suspension in an aqueous liquid, as a non-aqueous liquid, as an oil-in-water emulsion, or as a water-in-oil liquid emulsion. In addition to dosage forms described herein, the composition may be administered by controlled release means and/or delivery devices. The compositions can be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

In some embodiments, solid formulations comprising a composition as described herein is rectangular, tubular, oblong, circular, round, oval in shape. In some embodiments, the solid formulations comprising a composition as described herein is oblong in shape. In some embodiments, the solid formulations comprising the composition as described herein is oval in shape.

In some embodiments, compositions as described herein are administered as one or more soft gel capsules. In some embodiments, a dose is a single soft gel, two soft gels, three softgels, or more. In some embodiments, the soft gel capsules comprise an amount of composition as described herein of at least or about 50 mg, 100 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1500 mg, 2000 mg, or ranges including and/or spanning the aforementioned values. For example, the soft gel capsule may comprise the composition in an amount ranging from 300 mg to 400 mg, 250 mg to 500 mg, 350 mg to 400 mg, etc. In some embodiments, the soft gel capsules comprise a composition as described herein of about 400 mg. In some embodiments, the soft gel capsules about 100 to about 500 mg omega-3 fatty acids or esters thereof. In some embodiments, the soft gel capsules comprise about 50 mg to about 300 mg of maqui berry extract. In some embodiments, the soft gel capsules comprise about 2 to about 40 mg astaxanthin. In some embodiments, the soft gel capsules comprise about 1 to about 20 mg zexanthin. In some embodiments, the soft gel capsules comprise about 10 to about 50 mg lutein. In some embodiments, the soft gel capsules comprise about 20 mg to about 80 mg anthocyanins from maqui berry extract. In some embodiments, the soft gel capsules comprise one or more excipient ingredients. In some embodiments, the one or more excipient ingredients comprises one or more inert ingredients. In some embodiments, the inert ingredients may comprise at least one of fish oil, gelatin, glycerin, purified water, yellow beeswax, and lecithin oil.

In some embodiments, compositions as described herein are administered in one or more soft gel capsules, wherein the one or more soft gel capsules comprise a range of about 50% to about 95% omega-3 fatty acids or esters thereof, about 1% to about 50% maqui berry extract, and 1% to about 20% one or more carotenoids. In some embodiments, the soft gel capsules comprises at least or about 10%, 20%, 30%, 40%, 50%, 60%, 70%, 75%, 80%, 85%, 90%, 95% or more than 95% omega-3 fatty acids or esters thereof. In some embodiments, the soft gel capsules comprise one or more omega-3 fatty acids or esters of least or about 1%, 5%, 10%, 20%, 30%, 40%, 50%, or ranges including and/or spanning the aforementioned values. In some embodiments, the soft gel capsules comprise at least one or more carotenoids of about 1%, 2%, 3%, 4%, 5%, 6%, 7%, 8%, 9%, 10%, or ranges including and/or spanning the aforementioned values.[0061] The pharmaceutical compositions disclosed herein may be manufactured in a manner that is itself known, e.g., by means of conventional mixing, dissolving, granulating, dragee-making, levigating, emulsifying, encapsulating, entrapping or tableting processes. Additionally, the active ingredients are contained in an amount effective to achieve its intended purpose. Many of the compounds used in the pharmaceutical combinations disclosed herein may be provided as salts with pharmaceutically compatible counterions.

Multiple techniques of administering a compound, salt and/or composition exist in the art including, but not limited to, oral, rectal, pulmonary, topical, aerosol, injection, infusion and parenteral delivery, including intramuscular, subcutaneous, intravenous, intramedullary injections, intrathecal, direct intraventricular, intraperitoneal, intranasal and intraocular injections. In some embodiments, the composition, or a composition comprising pharmaceutically acceptable salts thereof, is administered orally.

One may also administer the compound(s), salt(s), and/or composition in a local rather than systemic manner, for example, via injection or implantation of the compound directly into the affected area, often in a depot or sustained release formulation. Furthermore, one may administer the compound in a targeted drug delivery system, for example, in a liposome coated with a tissue-specific antibody. The liposomes will be targeted to and taken up selectively by the organ. For example, intranasal or pulmonary delivery to target a respiratory disease or condition may be desirable.

The pharmaceutical compositions may contain one or more omega-3 fatty acids, a maqui berry extract, one or more carotenoids, in an amount effective for the desired therapeutic effect. In some embodiments, the pharmaceutical compositions are in a unit dosage form and comprise from about 0.1 mg or less to about 5000 mg or more per unit dosage form. In further embodiments, the pharmaceutical compositions comprise from about 1 to about 500 mg per unit dosage form or from about 500 to 5000 mg per unit dosage form. Such dosage forms may be solid, semisolid, liquid, an emulsion, or adapted for delivery by oral administration.

In some embodiments, the compositions as described herein may further comprise a carrier or a pharmaceutical carrier. In some embodiments, the carrier or pharmaceutical carrier is selected on the basis of compatibility with the composition disclosed herein, and the release profile properties of the desired dosage form. Exemplary carrier materials include, e.g., binders, suspending agents, disintegration agents, filling agents, surfactants, solubilizers, stabilizers, lubricants, wetting agents, diluents, and the like. Pharmaceutically compatible carrier materials include, but are not limited to, acacia, gelatin, colloidal silicon dioxide, calcium glycerophosphate, calcium lactate, maltodextrin, glycerine, magnesium silicate, polyvinylpyrrollidone (PVP), cholesterol, cholesterol esters, sodium caseinate, soy lecithin, taurocholic acid, phosphotidylcholine, sodium chloride, tricalcium phosphate, dipotassium phosphate, cellulose and cellulose conjugates, sugars sodium stearoyl lactylate, carrageenan, monoglyceride, diglyceride, pregelatinized starch, and the like. employed can be, for example, a solid or liquid. Examples of solid carriers include lactose, terra alba, sucrose, talc, gelatin, agar, pectin, acacia, magnesium stearate, and stearic acid. Examples of liquid carriers are sugar syrup, peanut oil, olive oil, lower alcohols, and water. Examples of gaseous carriers include carbon dioxide and nitrogen.

In some embodiments, the compositions may further comprise one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions, suitable salts include sodium chloride, potassium chloride, sodium thiosulfate, sodium bisulfite and ammonium sulfate.

In some embodiments, the composition may further comprise a sugar. Exemplary sugars include, but are not limited to, trehalose, sucrose, mannitol, maltose, or glucose.

In some embodiments, the composition may further comprise a diluent. Example diluents include, but are not limited to, a buffered saline solution. In certain instances, diluents increase bulk of the composition to facilitate compression or create sufficient bulk for homogenous blend for capsule filling. Such compounds can include e.g., lactose, starch, mannitol, sorbitol, dextrose, microcrystalline cellulose such as Avicel®, dibasic calcium phosphate, dicalcium phosphate dihydrate, tricalcium phosphate, calcium phosphate, anhydrous lactose, spray-dried lactose, pregelatinized starch, compressible sugar, such as Di-Pac® (Amstar), mannitol, hydroxypropylmethylcellulose, hydroxypropylmethylcellulose acetate stearate, sucrose-based diluents, confectioner's sugar, monobasic calcium sulfate monohydrate, calcium sulfate dihydrate, calcium lactate trihydrate, dextrates, hydrolyzed cereal solids, amylose, powdered cellulose, calcium carbonate, glycine, kaolin, mannitol, sodium chloride, inositol, bentonite, and the like.

In some embodiments, the composition as described herein may further comprise a disintegration agent. Disintegration agents facilitate the breakup or disintegration of a substance. The term “disintegrate” include both the dissolution and dispersion of the dosage form when contacted with gastrointestinal fluid. Examples of disintegration agents include, but not limited to, a starch, e.g., a natural starch such as corn starch or potato starch, a pregelatinized starch such as National 1551 or Amijel®, or sodium starch glycolate such as Promogel® or Explotab®, a cellulose such as a wood product, methylcrystalline cellulose, e.g., Avicel®, Avicel® PH101, Avicel® PH102, Avicel® PH105, Elcema® P100, Emcocel®, Vivacel®, Ming Tia®, and Solka-Floc®, methylcellulose, croscarmellose, or a cross-linked cellulose, such as cross-linked sodium carboxymethylcellulose (Ac-Di-Sol®), cross-linked carboxymethylcellulose, or cross-linked croscarmellose, a cross-linked starch such as sodium starch glycolate, a cross-linked polymer such as crospovidone, a cross-linked polyvinylpyrrolidone, alginate such as alginic acid or a salt of alginic acid such as sodium alginate, a clay such as Veegum® HV (magnesium aluminum silicate), a gum such as agar, guar, locust bean, Karaya, pectin, or tragacanth, sodium starch glycolate, bentonite, a natural sponge, a surfactant, a resin such as a cation-exchange resin, citrus pulp, sodium lauryl sulfate, sodium lauryl sulfate in combination starch, and the like.

In some embodiments, the composition as described herein may further comprise a filling agent. Filing agents include, but are not limited to, lactose, calcium carbonate, calcium phosphate, dibasic calcium phosphate, calcium sulfate, microcrystalline cellulose, cellulose powder, dextrose, dextrates, dextran, starches, pregelatinized starch, sucrose, xylitol, lactitol, mannitol, sorbitol, sodium chloride, polyethylene glycol, and the like.

In some embodiments, the composition as described herein may further comprise a lubricant or glidant. Exemplary lubricants include, but are not limited to, stearic acid, calcium hydroxide, talc, sodium stearyl fumerate, a hydrocarbon such as mineral oil, or hydrogenated vegetable oil such as hydrogenated soybean oil (Sterotex®), higher fatty acids and their alkali-metal and alkaline earth metal salts, such as aluminum, calcium, magnesium, zinc, stearic acid, sodium stearates, glycerol, talc, waxes, Stearowet®, boric acid, sodium benzoate, sodium acetate, sodium chloride, leucine, a polyethylene glycol (e.g., PEG-4000) or a methoxypolyethylene glycol such as Carbowax™ sodium oleate, sodium benzoate, glyceryl behenate, polyethylene glycol, magnesium or sodium lauryl sulfate, colloidal silica such as Syloid™, Cab-O—Si®, a starch such as corn starch, silicone oil, a surfactant, and the like.

In some embodiments, the composition as described herein may further comprise a plasticizer. Plasticizers include, but are not limited to, compounds used to soften the microencapsulation material or film coatings to make them less brittle. Suitable plasticizers include, e.g., polyethylene glycols such as PEG 300, PEG 400, PEG 600, PEG 1450, PEG 3350, and PEG 800, stearic acid, propylene glycol, oleic acid, triethyl cellulose and triacetin. Plasticizers can also function as dispersing agents or wetting agents.

In some embodiments, the composition as described herein may further comprise a stabilizer. Solubilizers include, but are not limited to, compounds such as triacetin, triethylcitrate, ethyl oleate, ethyl caprylate, sodium lauryl sulfate, sodium doccusate, vitamin E TPGS, dimethylacetamide, N-methylpyrrolidone, N-hydroxyethylpyrrolidone, polyvinylpyrrolidone, hydroxypropylmethyl cellulose, hydroxypropyl cyclodextrins, ethanol, n-butanol, isopropyl alcohol, cholesterol, bile salts, polyethylene glycol 200-600, glycofurol, transcutol, propylene glycol, and dimethyl isosorbide and the like.

In some embodiments, the composition as described herein may further comprise a stabilizer. Stabilizers include, but are not limited to, compounds such as any antioxidation agents, buffers, acids, preservatives and the like. Exemplary stabilizers include L-arginine hydrochloride, tromethamine, albumin (human), citric acid, benzyl alcohol, phenol, disodium biphosphate dehydrate, propylene glycol, metacresol or m-cresol, zinc acetate, polysorbate-20 or Tween® 20, or trometamol.

In some embodiments, the composition as described herein may further comprise a suspending agent. Suspending agents include, but are not limited to, compounds such as polyvinylpyrrolidone, e.g., polyvinylpyrrolidone K12, polyvinylpyrrolidone K17, polyvinylpyrrolidone K25, or polyvinylpyrrolidone K30, vinyl pyrrolidone/vinyl acetate copolymer (S630), polyethylene glycol, e.g., the polyethylene glycol can have a molecular weight of about 300 to about 6000, or about 3350 to about 4000, or about 7000 to about 5400, sodium carboxymethylcellulose, methylcellulose, hydroxypropylmethylcellulose, hydroxymethylcellulose acetate stearate, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, poly sorbate-80, sodium alginate, polyethoxylated sorbitan monolaurate, polyethoxylated sorbitan monolaurate, povidone and the like.

In some embodiments, the composition as described herein may further comprise a surfactant. Surfactants include, but are not limited to, compounds such as sodium lauryl sulfate, sodium docusate, Tween 60 or 80, triacetin, vitamin E TPGS, sorbitan monooleate, polyoxyethylene sorbitan monooleate, polysorbates, polaxomers, bile salts, glyceryl monostearate, copolymers of ethylene oxide and propylene oxide, e.g., Pluronic® (BASF), and the like. Additional surfactants include polyoxyethylene fatty acid glycerides and vegetable oils, e.g., polyoxyethylene (60) hydrogenated castor oil, and polyoxyethylene alkylethers and alkylphenyl ethers, e.g., octoxynol 10, octoxynol 40. Sometimes, surfactants is included to enhance physical stability or for other purposes.

In some embodiments, the composition as described herein may further comprise a viscosity enhancing agent. Viscosity enhancing agents include, but are not limited to, methyl cellulose, xanthan gum, carboxymethyl cellulose, hydroxypropyl cellulose, hydroxypropylmethyl cellulose, hydroxypropylmethyl cellulose acetate stearate, hydroxypropylmethyl cellulose phthalate, carbomer, polyvinyl alcohol, alginates, acacia, chitosans and combinations thereof.

In some embodiments, the composition as described herein may further comprise a wetting agent. Wetting agents include, but are not limited to, compounds such as oleic acid, glyceryl monostearate, sorbitan monooleate, sorbitan monolaurate, triethanolamine oleate, polyoxyethylene sorbitan monooleate, polyoxyethylene sorbitan monolaurate, sodium docusate, sodium oleate, sodium lauryl sulfate, sodium doccusate, triacetin, Tween 80, vitamin E TPGS, ammonium salts and the like.

Pharmaceutical compositions provided herein may be suitable for injectable use and may include sterile aqueous solutions or dispersions. Furthermore, the compositions can be in the form of sterile powders for the extemporaneous preparation of such sterile injectable solutions or dispersions. In some embodiments, the pharmaceutical compositions may be stable under the conditions of manufacture and storage. In some embodiments, the sterile aqueous solution or dispersions may further comprise a carrier. In some embodiments, the carrier can be a solvent or dispersion medium containing, for example, water, ethanol, polyol (e.g., glycerol, propylene glycol and liquid polyethylene glycol), vegetable oils, and suitable mixtures thereof.

Contemplated herein are pharmaceutical compositions including one or more omega-3 fatty acids, a maqui berry extract, one or more carotenoids in combination with at least one additional active agent. The pharmaceutical composition described herein and the at least one additional active agent(s) may be present in a single formulation or in multiple formulations provided together, or may be unformulated. In some embodiments, the pharmaceutical compositions can be administered with one or more additional agents together in a single composition. For example, a composition including one or more omega-3 fatty acids, a maqui berry extract, one or more carotenoids can be administered in one composition, and at least one of the additional agents can be administered in a second composition. In a further embodiment, a composition including one or more omega-3 fatty acids, a maqui berry extract, one or more carotenoids are co-packaged in a kit. For example, a drug manufacturer, a drug reseller, a physician, a compounding shop, or a pharmacist can provide a kit comprising a disclosed compound or product and another component for delivery to a patient.

### Foodstuffs

Foodstuffs and other comestibles including a composition described herein are provided, wherein an amount of the composition in the foodstuff has been fortified (e.g., enriched or concentrated). In some embodiments, a composition described herein may be added to foodstuffs for consumption by a subject. In some embodiments, the composition may be integrated into one or more ingredients of a foodstuff. In some embodiments, the composition may be prepared as an ingredient, or may be unprepared. The composition, or preparation including the compound, may be added prior to preparation, during preparation, or following preparation. Preparation may without limitation include cooking, mixing, flavoring, seasoning, blending, boiling, frying, baking, or other processes known in the art. Fortification may be at a level so as to provide a therapeutic daily dosage of the composition as described elsewhere herein; however, beneficial effects may also be obtained at amounts below such dosages.

A composition comprising one or more omega-3 fatty acids, a maqui berry extract, one or more carotenoids, as provided herein may be present as a constituency in foodstuffs by operation of processes known. By way of example, the composition may be present in the foodstuff in a concentration of at least about 1%, at least about 2%, at least about 3%, at least about 4%, at least about 5%, at least about 6%, at least about 7%, at least about 8%, at least about 9%, at least about 10%, at least about 20%, at least about 30%, at least about 40%, at least about 50%, or higher, for example, 1% to 2% or 3% or 4% or 5% or 6% or 7% or 8% or 9% or 10% or 20% or 30% or 40% or 50%. In some embodiments, the composition may be administered with a foodstuff; for example, containing 1 to 2000 mg, 1 to 1000 mg, 1 to 500 mg, 10 to 100 mg, 5 to 50 mg, or any amount in between of the composition.

### Methods of Use

Some embodiments disclosed herein relate to a method of preventing, treating, and/or ameliorating a condition (e.g., of the eye) that can include administering to a subject a therapeutically effective amount of one or more compounds, combinations, or compositions as described herein. In some embodiments, the method may comprise administering a composition comprising one or more of DHA omega-3 fatty acid, lutein, zeaxanthin, astaxanthin, and/or an anthocyanin, pharmaceutically acceptable salt(s) of any one thereof. In some embodiments, the method may comprise administering a pharmaceutical composition that includes one or more of the compounds described herein. In some embodiments, a dose or doses of the compound(s) or a composition (e.g., a pharmaceutical composition) are administered to the subject.

In some embodiments, a suitable dose will often be in the range of from about 0.05 mg/kg to about 10 mg/kg. For example, a suitable dose may be in the range from about 0.10 mg/kg to about 7.5 mg/kg of body weight per day, such as about 0.15 mg/kg to about 5.0 mg/kg of body weight of the recipient per day, about 0.2 mg/kg to 4.0 mg/kg of body weight of the recipient per day, or any amount in between. The compound may be administered in unit dosage form; for example, containing 1 to 2000 mg, 1 to 1000 mg, 1 to 500 mg, 10 to 100 mg, 5 to 50 mg, or any amount in between, of active ingredient per unit dosage form. The desired dose may conveniently be presented in a single dose (e.g., once daily) or as divided doses administered at appropriate intervals, for example, as two, three, four or more sub-doses per day. The sub-dose itself may be further divided, e.g., into a number of discrete loosely spaced administrations.

In some embodiments, the compositions as described herein may be administered daily. In some embodiments, at least or about 1, 2, 3, 4, 5, 6, 7, 8, 9, 10, or more than 10 doses of the compositions as described herein is provided.

In some embodiments, compounds as disclosed herein may be administered simultaneously or sequentially. In some embodiments, where the compounds are not provided in the same composition (e.g., multiple separate compositions are provided to deliver the appropriate combination), the compositions may be administered via different routes (e.g., oral and intravenously, etc.). In other embodiments, the composition is administered to the subject concurrent with the subject receiving one or more additional therapeutic agents (e.g., approved eye care drugs, etc.). In some embodiments, concurrent administration can be effected by multiple administrations within a limited period of time (for example, a dosage forms taken together) or concurrent administration can be effected simultaneously or near simultaneously.

In some embodiments, the disease or disorder is an ocular disease or disorder. For example, the disease or disorder is a degenerative disease, cancer, cardiovascular disease, diabetes, or arthritis. Exemplary ocular diseases or disorder include, but are not limited to, inflammatory conjunctivitis, including allergic and giant papillary conjunctivitis, macular edema, uveitis, endophthalmitis, scleritis, corneal ulcers, dry eye syndrome, glaucoma, ischemic retinal disease, corneal transplant rejection, complications related to intraocular surgery such intraocular lens implantation and inflammation associated with cataract surgery, Behcet's disease, Stargardt disease, immune complex vasculitis, Fuch's disease, Vogt-Koyanagi-Harada disease, subretinal fibrosis, keratitis, vitreo-retinal inflammation, ocular parasitic infestation/migration, retinitis pigmentosa, cytomeglavirus retinitis and choroidal inflammation. In some embodiments, the ocular diseases or disorders that may be amendable to treatment by the methods and compositions provided herein may include, without limitation, ectropion, lagophthalmos, blepharochalasis, ptosis, xanthelasma of the eyelid, parasitic infestation of the eyelid, dermatitis of the eyelid, dacryoadenitis, epiphora, dysthyroid exophthalmos, conjunctivitis, scleritis, keratitis, corneal ulcer, corneal abrasion, snow blindness, arc eye, Thygeson's superficial punctate keratopathy, corneal neovascularization, Fuchs' dystrophy, keratoconus, keratoconjunctivitis sicca, iritis, uveitis, sympathetic ophthalmia, cataracts, chorioretinal inflammation, focal chorioretinal inflammation, focal chorioretinitis, focal choroiditis, focal retinitis, focal retinochoroiditis, disseminated chorioretinal inflammation, disseminated chorioretinitis, disseminated choroiditis, disseminated retinitis, disseminated retinochoroiditis, exudative retinopathy, posterior cyclitis, pars planitis, Harada's disease, chorioretinal scars, macula scars of posterior pole, solar retinopathy, choroidal degeneration, choroidal atrophy, choroidal sclerosis, angioid streaks, hereditary choroidal dystrophy, choroideremia, choroidal dystrophy (central arealor), gyrate atrophy (choroid), ornithinaemia, choroidal haemorrhage and rupture, choroidal haemorrhage (not otherwise specified), choroidal haemorrhage (expulsive), choroidal detachment, retinoschisis, retinal artery occlusion, retinal vein occlusion, hypertensive retinopathy, diabetic retinopathy, retinopathy, retinopathy of prematurity, macular degeneration, Bull's Eye maculopathy, epiretinal membrane, peripheral retinal degeneration, hereditary retinal dystrophy, retinitis pigmentosa, retinal haemorrhage, separation of retinal layers, central serous retinopathy, retinal detachment, macular edema, glaucoma—optic neuropathy, glaucoma suspect—ocular hypertension, primary open-angle glaucoma, primary angle-closure glaucoma, floaters, Leber's hereditary optic neuropathy, optic disc drusen, strabismus, ophthalmoparesis, progressive external ophthaloplegia, esotropia, exotropia, disorders of refraction and accommodation, hypermetropia, myopia, astigmastism, anisometropia, presbyopia, internal ophthalmoplegia, amblyopia, Leber's congenital amaurosis, scotoma, anopsia, color blindness, achromatopsia, maskun, nyctalopia, blindness, River blindness, micropthalmia, coloboma, red eye, Argyll Robertson pupil, keratomycosis, xerophthalmia, aniridia, sickle cell retinopathy, ocular neovascularization, retinal neovascularization, subretinal neovascularization; rubeosis iritis inflammatory diseases, chronic posterior and pan uveitis, neoplasms, retinoblastoma, pseudoglioma, neovascular glaucoma; neovascularization resulting following a combined vitrectomy-2 and lensectomy, vascular diseases, retinal ischemia, choroidal vascular insufficiency, choroidal thrombosis, neovascularization of the optic nerve, diabetic macular edema, cystoid macular edema, proliferative vitreoretinopathy, and neovascularization due to penetration of the eye or ocular injury. In some embodiments, the ocular disease or disorder is selected from a group consisting of allergies, glaucoma, cataract, corneal disease, vitreo-retinal diseases, optic nerve diseases or disorders, oculosystemic diseases and disorders, uvea diseases or disorders, or diabetic eye disease. In some embodiments, the ocular disease or disorder is Meibomian gland inflammation or Meibomian gland dysfunction. In some embodiments, the ocular disease or disorder is dry eye disease. In some embodiments, the disease or disorder is characterized by inflammation.

In some embodiments, administration of the compositions disclosed herein result in an improvement in eye health and/or function. In some embodiments, the improvement in eye health is measured as a change in subjective symptoms of dry eye. In some embodiments, a score of 0-3 is assigned to the common symptoms of dry eye such as itching or burning, sandy or gritty sensation, redness, blurring of vision, ocular fatigue and/or excessive blinking, respectively. When absent (0), sometimes present (1), frequently present (2), and always present (3). (Score of 0-6 was mild, 6.1-12 moderate and 12.1-18, severe dry eye). In some embodiments, treatment results in a lowering in symptom score (for any of the aforementioned symptoms) of greater than or at least about: 2 points, 3 points, 4 points, 5 points, 6 points, 8 points, or ranges including and/or spanning the aforementioned values.

In some embodiments, improvements in eye health or function are measured using a Schirmer's I test for tear production, tear film break-up test (TBUT), the Rose Bengal score (RBS) as a measure of ocular surface integrity, and conjunctival impression cytology (CIC) scores for cellular changes and goblet cell density. In some embodiments, each subject may undergo an ocular examination, which may include measurement of best corrected visual acuity (BCVA) and slit lamp biomicroscopy. In some embodiments, slit lamp examination includes assessment of the lid margins, eyelashes and meibomian gland orifice for any blockage or stenosis. In some embodiments, platelet count, prothrombin time and activate partial thromboplastin time (APTT) can be measured. In some embodiments, the improvement in function is measured as one or more of an increase in tear flow from the lacrimal glands, improvement in the quality of the lipid secretion from the meibomian glands, improvement of tear breakup time, and/or augmentation of DHA fatty acid in the retina (e.g., a retina damaged by blue light). In some embodiments, function of the meibomian gland is improved. In some embodiments, after 30 days or 90 days of one or more of these measures of eye health improves (symptoms lowers or indications/markers of health increase) by greater than or at least about: 10%, 25%, 50%, 75%, 85%, 95%, 100%, or ranges including and/or spanning the aforementioned values.

In some embodiments, one or more doses comprising a compound or composition as described herein is administered to the subject over a period of time. In some embodiments, the period of time is 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, or more than 12 weeks. The period of time, in some embodiments, is about 1 month, 2 months, 3 months, 4 months, 5 months, 6 months, 7 months, 8 months, 9 months, 10 months, 11 months, 1 year, 2 years, 3 years, 4 years, 5 years, 6 years, 7 years, 8 years, or more than 8 years. In some embodiments, the plurality of doses comprising a compound or composition as described herein is administered chronically. In some embodiments, one or more doses comprising a compound or composition as described herein is administered to the subject daily for a period of time.

Methods as described herein, in some embodiments comprise providing a dose a composition as described herein at least 1, 2, 3, 4, 5, 6, 7, 8, 9, 10, 11, 12, more than 12 times a day. In some embodiments, a dose of a composition as described herein is administered at least 4 times a day. In some embodiments, a dose of a composition as described herein is administered about once times a day. In some embodiments, a dose of a composition as described herein is administered about 2 times a day. In some embodiments, a dose of a composition as described herein is administered about 3 times a day. In some embodiments, a dose of a composition as described herein is administered about 4 times a day. Administration, in some embodiments, is about every 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, or 24 hours. In some embodiments, a time between administration is at least or about 1 hour, 2 hours, 3 hours, 4 hours, 5 hours, 6 hours, 7 hours, 8 hours, 9 hours, 10 hours, 11 hours, 12 hours, or more than 12 hours. In some embodiments, a time between administration is in a range of 0 hours to 24 hours, 1 hour to 23 hours, 2 hours to 22 hours, 3 hours to 21 hours, 4 hours to 20 hours, 5 hours to 19 hours, 6 hours to 18 hours, 7 hours to 17 hours, 8 hours to 16 hours, 9 hours to 15 hours, and 10 hours to 12 hours.

### Kits

According to another aspect of the disclosure, kits are provided. Kits, according to the present disclosure, include package(s) or containers comprising the compositions disclosed herein. The kit may further include an instruction letter or package-associated instruction for the treatment and/or prophylaxis of a medical condition. The phrase “package” means any vessel containing the compositions (including stem cells, media, and/or media supplement) presented herein. For example, the package can be a box or wrapping. Kits may optionally contain instructions for administering compositions of the present disclosure to a subject having a condition in need of treatment. Kits may also comprise instructions for approved uses of compounds herein by regulatory agencies, such as the United States Food and Drug Administration. Kits may optionally contain labeling or product inserts for the present compositions. The package(s) and/or any product insert(s) may themselves be approved by regulatory agencies.

In some embodiments, the kit comprises a composition as described herein in a sterilized vessel. In some embodiments, the sterilized vessel may comprise a plastic or glass container of various sizes and capacities. The sterilized vessel may optionally contain a composition described herein with a volume of e.g., 50 ml, 100 ml, 150 ml, 250 ml, 300 ml, 500 ml, 1000 ml, or ranges including and/or spanning the aforementioned values. Sterilized vessels enable maintain sterility of the compositions, facilitate transportation and storage, and allow administration of the composition without a prior sterilization step.

The present disclosure also provides a kit for administering a composition described herein to a subject in need thereof, comprising: at least one of omega-3 fatty acid. In one embodiment, a kit comprises a composition comprising omega-3 fatty acid, astaxanthin, zeaxanthin, lutein, and maqui berry fruit extract, or a combination thereof. In another embodiment, a kit comprises a composition further comprising fish oil, gelatin, glycerin, purified water, yellow beeswax, lecithin oil, or a combination thereof. In some embodiments, the kit comprises a soft gel cap as described herein and a sterile container.

The kit may further comprise one or more syringes and/or syringe needles for injection the pharmaceutical formulation to the patient. For the viscous liquid formulation, syringe needles with high fluidic capacity, such as DEPOTONE syringe needles (Imprint Pharmaceuticals Ltd., UK).

The kit may optionally further include instructions. The instruction may also describe how to administer the resulting pharmaceutical formulation to a patient. It is noted that the instructions may optionally describe the administration methods according to the present disclosure.

### EXAMPLES

Additional embodiments are disclosed in further detail in the following examples, which are not in any way intended to limit the scope of the claims. One skilled in the art can easily modify or change the formulation within the specific description to provide a unique desired product which falls within the scope of the claims.

### Example 1

The following Table provides one embodiment of a composition as disclosed herein. As shown, the composition may comprise one or more omega-3 fatty acid, one or more carotenoids, and maqui berry extract.

TABLE 1
Amount per serving

| | Potency | |
|---|---|---|
| Total Omega-3 fatty acid (including DHA, EPA, DPA) | 250 | mg |
| Total carotenoids | 11 | mg |
| Maqui Berry extract (standardized to 10% anthocyanins) | 100 | mg |

One composition containing the ingredients specified in Table 1 above, is administered to a person suffering from one or more eye condition or disease.

### Example 2

The following Table provides one embodiment of a composition as disclosed herein. As shown, the composition may comprise one or more omega-3 fatty acid, lutein, one or more carotenoids, and maqui berry extract.

TABLE 2
Amount per serving

| | Potency | |
|---|---|---|
| Total Omega-3 fatty acid (including DHA, EPA, DPA) | 250 | mg |
| Lutein | 50 | mg |
| Total carotenoids | 11 | mg |
| Maqui Berry extract (standardized to 10% anthocyanins) | 100 | mg |

One composition containing the ingredients specified in Table 2 above, is administered to a person suffering from one or more eye condition or disease.

### Example 3

The following Table provides one embodiment of a composition as disclosed herein. As shown, the composition may comprise each of DHA containing omega-3 fatty acid, lutein, zeaxanthin, astaxanthin, and maqui berry extract.

TABLE 3
Amount per serving

| | Potency | |
|---|---|---|
| Total Omega-3 fatty acid (including DHA, EPA, DPA) | 125 | mg |
| DHA Omega-3 fatty acid | 100 | mg |
| Lutein | 12.5 | mg |
| Zeaxanthin | 2.5 | mg |
| Astaxanthin | 3 | mg |
| Maqui Berry extract (standardized to 10% anthocyanins) | 50 | mg |

One composition containing the ingredients specified in Table 3 above, is administered daily to a person suffering from dry eye. After 30 days, aqueous tear flow increases as demonstrated by improvement in the Schirmer test. Improvement of the tear film over the cornea is demonstrated by an increase in tear breakup time (TBUT) score. Visual observation finds a reduction in blocked meibomian ducts and a decrease in inflammation.

### Example 4

The following Table provides one embodiment of a composition as disclosed herein. As shown, the composition may comprise each of DHA containing omega-3 fatty acid, lutein, zeaxanthin, astaxanthin, and maqui berry extract.

TABLE 4
Amount per serving

| | Potency | |
|---|---|---|
| Total Omega-3 fatty acid (including DHA, EPA, DPA) | 250 | mg |
| DHA Omega-3 fatty acid | 200 | mg |
| Lutein | 25 | mg |
| Zeaxanthin | 5 | mg |
| Astaxanthin | 6 | mg |
| Maqui Berry extract (standardized to 10% anthocyanins) | 100 | mg |

One softgel containing the ingredients specified in Table 4 above, is administered daily to a person suffering from dry eye. After 30 days, aqueous tear flow increases as demonstrated by improvement in the Schirmer test. Improvement of the tear film over the cornea is demonstrated by an increase in tear breakup time (TBUT) score. Visual observation finds a reduction in blocked meibomian ducts and a decrease in inflammation.

### Example 5

The following Table provides one embodiment of a composition as disclosed herein. As shown, the composition may comprise each of DHA containing omega-3 fatty acid, lutein, zeaxanthin, astaxanthin, and maqui berry extract.

TABLE 5
Amount per serving

| | Potency | |
|---|---|---|
| Total Omega-3 fatty acid (including DHA, EPA, DPA) | 500 | mg |
| DHA Omega-3 fatty acid | 400 | mg |
| Lutein | 50 | mg |
| Zeaxanthin | 10 | mg |
| Astaxanthin | 12 | mg |
| Maqui Berry extract (standardized to 10% anthocyanins) | 200 | mg |

One softgel containing the ingredients specified in Table 5 above, is administered daily to a person suffering from dry eye. After 30 days, aqueous tear flow increases as demonstrated by improvement in the Schirmer test. Improvement of the tear film over the cornea is demonstrated by an increase in tear breakup time (TBUT) score. Visual observation finds a reduction in blocked meibomian ducts and a decrease in inflammation.

### Example 6

The following Table provides one embodiment of a composition as disclosed herein. As shown, the composition may comprise one or more omega-3 fatty acid, one or more carotenoids, maqui berry extract and one or more excipient(s).

TABLE 6
Amount per serving

| | Potency (w/w) |
|---|---|
| Total Omega-3 fatty acid (including DHA, EPA, DPA) | 64% |
| Total carotenoids | 2% |
| Maqui Berry extract (standardized to 10% anthocyanins) | 26% |
| One or more excipient(s) | 8% |

One composition containing the ingredients specified in Table 6 above, is administered to a person suffering from one or more eye condition or disease.

### Example 7

The following Table provides one embodiment of a composition as disclosed herein. As shown, the composition may comprise one or more omega-3 fatty acid, lutein, one or more carotenoids, maqui berry extract and one or more excipient(s).

TABLE 7
Amount per serving

| | Potency (w/w) |
|---|---|
| Total Omega-3 fatty acid (including DHA, EPA, DPA) | 64% |
| DHA Omega-3 fatty acid | 80% |
| Lutein | 6% |
| Total carotenoids | 2% |
| Maqui Berry extract (standardized to 10% anthocyanins) | 26% |
| One or more excipient | 2% |

One composition containing the ingredients specified in Table 7 above, is administered to a person suffering from one or more eye condition or disease.

### Example 8

The following Table provides one embodiment of a composition as disclosed herein. As shown, the composition may comprise each of DHA containing omega-3 fatty acid, lutein, zeaxanthin, astaxanthin, and maqui berry extract.

TABLE 8
Amount per serving

| | Potency (w/w) |
|---|---|
| Total Omega-3 fatty acid (including DHA, EPA, DPA) | 64% |
| Lutein | 6% |
| Zeaxanthin | 1% |
| Astaxanthin | 1% |
| Maqui Berry extract (standardized to 10% anthocyanins) | 26% |
| One or more excipient | 2% |

One softgel containing the ingredients specified in Table 8 above, is administered daily to a person suffering from dry eye. After 30 days, aqueous tear flow increases as demonstrated by improvement in the Schirmer test. Improvement of the tear film over the cornea is demonstrated by an increase in tear breakup time (TBUT) score. Visual observation finds a reduction in blocked meibomian ducts and a decrease in inflammation.

### Example 9

The following Table provides one embodiment of a composition as disclosed herein. As shown, the composition may comprise each of DHA containing omega-3 fatty acid, lutein, zeaxanthin, astaxanthin, maqui berry extract, and one or more excipient(s). The one or more excipient is selected from fish oil, gelatin, glycerin, purified water, yellow beeswax, lecithin oil, or a combination thereof

TABLE 9
Amount per serving

| | Potency (w/w) |
|---|---|
| Total Omega-3 fatty acid (including DHA, EPA, DPA) | 64% |
| DHA Omega-3 fatty acid | 80% |
| Lutein | 6% |
| Zeaxanthin | 1% |
| Astaxanthin | 1% |
| Maqui Berry extract (standardized to 10% anthocyanins) | 26% |
| One or more excipient | 2% |

One softgel containing the ingredients specified in Table 9 above, is administered daily to a person suffering from dry eye. After 30 days, aqueous tear flow increases as demonstrated by improvement in the Schirmer test. Improvement of the tear film over the cornea is demonstrated by an increase in tear breakup time (TBUT) score. Visual observation finds a reduction in blocked meibomian ducts and a decrease in inflammation.

### Example 10

The following Table provides one embodiment of a composition as disclosed herein. As shown, the composition may comprise each of DHA containing omega-3 fatty acid, lutein, zeaxanthin, astaxanthin, and maqui berry extract.

TABLE 10
Amount per serving

| | Potency (w/w) |
|---|---|
| Total Omega-3 fatty acid (including DHA, EPA, DPA) | 60 to 70% |
| Lutein | 5 to 10% |
| Zeaxanthin | 0.5 to 2.5% |
| Astaxanthin | 0.5 to 2.5% |
| Maqui Berry extract (standardized to 10% anthocyanins) | 20 to 30% |
| One or more excipient | 0 to 10% |

One composition containing the ingredients specified in Table 10 above, is administered daily to a person suffering from dry eye. After 30 days, aqueous tear flow increases as demonstrated by improvement in the Schirmer test. Improvement of the tear film over the cornea is demonstrated by an increase in tear breakup time (TBUT) score. Visual observation finds a reduction in blocked meibomian ducts and a decrease in inflammation.

### Example 11

The following Table provides one embodiment of a composition as disclosed herein. As shown, the composition may comprise each of DHA containing omega-3 fatty acid, lutein, zeaxanthin, astaxanthin, maqui berry extract, and one or more extract.

TABLE 11
Amount per serving

| | Potency (w/w) |
|---|---|
| Total Omega-3 fatty acid (including DHA, EPA, DPA) | 64% |
| DHA Omega-3 fatty acid | 80% |
| Lutein | 6% |
| Zeaxanthin | 1% |
| Astaxanthin | 1% |
| Maqui Berry extract (standardized to 10% anthocyanins) | 26% |
| One or more excipient(s) | 2% |

One softgel containing the ingredients specified in Table 11 above, is administered daily to a person suffering from dry eye. After 30 days, aqueous tear flow increases as demonstrated by improvement in the Schirmer test. Improvement of the tear film over the cornea is demonstrated by an increase in tear breakup time (TBUT) score. Visual observation finds a reduction in blocked meibomian ducts and a decrease in inflammation.

### Example 12

The following Table provides one embodiment of a composition as disclosed herein. As shown, the composition may comprise each of DHA containing omega-3 fatty acid, lutein, zeaxanthin, astaxanthin, and maqui berry extract.

TABLE 12
Amount per serving

| | Potency (w/w) |
|---|---|
| Total Omega-3 fatty acid (including DHA, EPA, DPA) | 64% |
| Lutein | 6% |
| Zeaxanthin | 1% |
| Astaxanthin | 1% |
| Maqui Berry extract (standardized to 10% anthocyanins) | 26% |
| Fish oil, gelatin, glycerin, purified water, | 2% |
| yellow beeswax, lecithin oil, or a combination thereof | |

One softgel containing the ingredients specified in Table 12 above, is administered daily to a person suffering from dry eye. After 30 days, aqueous tear flow increases as demonstrated by improvement in the Schirmer test. Improvement of the tear film over the cornea is demonstrated by an increase in tear breakup time (TBUT) score. Visual observation finds a reduction in blocked meibomian ducts and a decrease in inflammation.

### Example 13

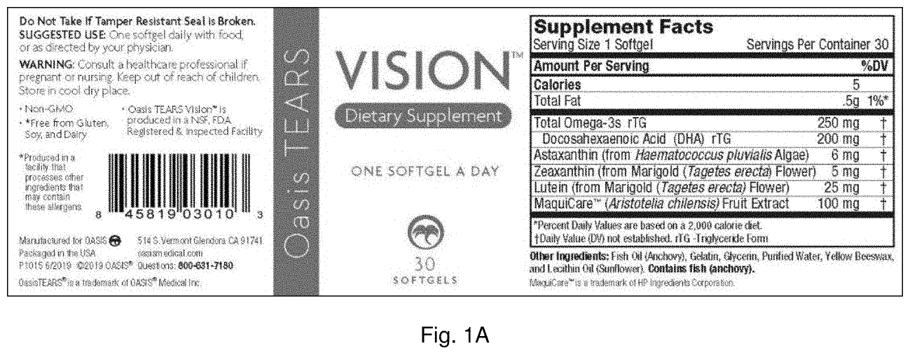

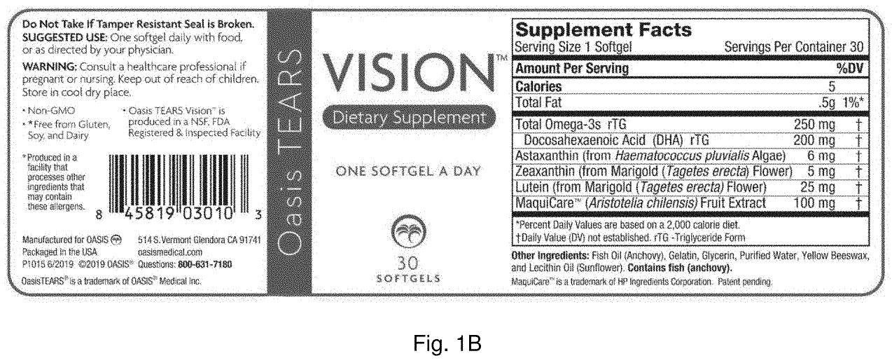

This is a prophetic example. A group of 50 patients suffering from chronic dry eye is separated into five groups of ten patients each: EXPT1, EXPT2, EXPT3, EXPT4, and CONT1. The EXPT1 subjects are given a once daily softgel containing the ingredients specified in FIG. 1A. The EXPT2 subjects are given a once daily softgel containing identical ingredients as in the softgel of FIG. 1A, but lacking an omega-3 fatty acid. The EXPT3 subjects are given a once daily softgel containing identical ingredients as in the softgel of FIG. 1A, but lacking the maqui berry extract. The EXPT4 subjects are given a once daily softgel containing identical ingredients as in the softgel of FIG. 1A, but lacking the carotenoids. The CONT1 subjects are given a placebo softgel comprising corn oil.

After 30 days, for each of the EXPT1-4 groups, a statistically significant improvement in aqueous tear flow is achieved compared to the CONT1 group, as demonstrated by improvement in the Schirmer test. A statistically significant improvement for the EXPT1-4 groups is also noted for improvement of the tear film over the cornea, as demonstrated by an increase in tear breakup time (TBUT) score. Visual observation of the EXPT1-4 groups finds a reduction in blocked meibomian ducts and a decrease in inflammation in eye. It is noted that the EXPT1 group achieves a statistically significant improvement over the EXPT2-4 groups for eye health. The combination of ingredients is noted as working synergistically to improve eye health and eye health test scores.

EXPT4 showed improvement over CONT1 by less damage to the retina due to blue light as measured by a decrease in inflammation, improved contrast sensitivity and improved visual acuity due to improved contrast.

EXPT3 showed improvement over CONT1 by improved tear flow as measured by the Schirmer test, and a reduction of ciliary muscle stress as measured by less eye fatigue, less headaches, and less shoulder and neck aches.

EXPT2 showed improvement over CONT 1 by improved retinal function due to the increased DHA present to protect against damage from blue light and oxidative stress. There was also an improvement in the lipid layer of the tear film resulting as measured by ocular microscopy and tear breakup time tests, etc.

EXPT1 showed improvement over CONT1 in all the measures stated above.

What is claimed is:

1. A composition for improving eye function, comprising:
one or more omega-3 fatty acids;
a maqui berry extract; and
one or more carotenoids.

2. The composition of claim 1, wherein the one or more carotenoids comprises lutein, zeaxanthin, and astaxanthin.

3. The composition of claim 1, wherein the one or more omega-3 fatty acids is present in the composition in an amount ranging from 100 mg to 3000 mg.

4. The composition of claim 1, wherein the omega-3 fatty acid comprises one or more of alpha-linolenic acid (ALA), docosapentaenic acid (DPA), or eicosapentaenoic acid (EPA).

5. The composition of claim 1, wherein the one or more omega-3 fatty acids has been enriched for DHA.

6. The composition of claim 1, wherein the DHA is present in the composition in an amount ranging from 100 mg to 3000 mg.

7. The composition of claim 1, wherein maqui berry extract is present in an amount ranging from 10 mg to 400 mg.

8. The composition of claim 2, wherein lutein is present in an amount ranging from 6 mg to 80 mg.

9. The composition of claim 2, wherein zeaxanthin is present in an amount ranging from 0.5 mg to 40 mg.

10. The composition of claim 2, wherein the astaxanthin is present in an amount ranging from 2 mg to 40 mg.

11. The composition of claim 1, wherein the maqui berry extract is standardized to less than 35% anthocyanins.

12. The composition of claim 1, wherein the combination of one or more omega-3 fatty acids, maqui berry extract, and one or more carotenoids is provided in an effective amount.

13. The composition of claim 1, wherein the each of the one or more omega-3 fatty acids, DHA, maqui berry extract, lutein, zeaxanthin, and astaxanthin is provided in an effective amount.

14. The composition of claim 1, wherein the composition is formulated for oral ingestion.

15. The composition of claim 1, wherein the composition is provided as a soft gel capsule.

16. A method of preventing, ameliorating, or treating an eye condition comprising:
administering composition of claim 1 to a subject at risk for or suffering from an eye condition.

17. The method of claim 16, wherein the eye condition comprises macular degeneration.

18. The method of claim 16, wherein the eye condition comprises dry eye.

19. The method of claim 16, wherein the eye condition is caused by environmental stress to the eye.

20. A method of preparing the composition of claim 1, comprising:
mixing one or more omega-3 fatty acids with a maqui berry extract and one or more carotenoids to provide a mixture of ingredients.

21. The method of claim 20, further comprising adding the mixture of ingredients to a soft gel.

22. An orally ingested composition for improving the functioning of an environmentally stressed eye comprising:
effective amounts of omega-3 fatty acids, maqui berry extract, lutein, zeaxanthin, and astaxanthin.

23. The orally ingested composition of claim 22, wherein the composition comprises:
100-3000 mg DHA omega-3 fatty acids, 10-400 mg maqui berry extract, 6-80 mg lutein, 0.5-40 mg zeaxanthin, and 2-40 mg astaxanthin.
